(19) United States
(12) Patent Application Publication
(10) Pub. No.: US 2007/0161957 A1
(43) Pub. Date: Jul. 12, 2007

(54) Hysteroscope Seal

(76) Inventors: Kevin V. Guenther, Minnetonka, MN (US); Kevin R. Arnal, Minnetonka, MN (US)

Correspondence Address: AMS RESEARCH CORPORATION, 10700 BREN ROAD WEST, MINNETONKA, MN, 55343, US

(21) Appl. No.: 11/619,897

(22) Filed: Jan. 4, 2007

Related U.S. Application Data
(60) Provisional application No. 60/757,085, filed on Jan. 6, 2006.

(51) Int. Cl. A61M 5/178

(52) U.S. Cl. 604/167.01

(57) ABSTRACT

A scope seal and method for sealing a working channel of a hysterscope is disclosed. The scope seal includes a proximal end through which a catheter can be received, a distal end that communicates with a fitting on the hysteroscope, and a substantially hollow body longitudinally extending between the proximal end and the distal end. The scope seal also includes a catheter sealing mechanism positionable within the body, a shaft seal, a chamber configured to receive the fitting of the hysteroscope and a pressure relief chamber. The catheter sealing mechanism can be any one of a duckbill valve, a conic valve, and/or a membrane. The chamber for receiving the fitting on the hysteroscope communicates with the fitting positioned on a proximal end of the hysteroscope, where the working channel of the hysteroscope accommodates a catheter therethrough in order to perform in vivo procedures.

### CROSS REFERENCE TO RELATED APPLICATION

This application is based on and claims the benefit of U.S. Provisional Application No. 60/757,085, filed Jan. 6, 2006, the entirety of which is hereby incorporated by reference.

### BACKGROUND OF THE INVENTION

1. Field of the Invention

The present invention relates to medical devices for performing procedures in vivo. The present invention particularly relates to seals for medical devices and, more particularly, to a seal for the working channel of a hysteroscope.

2. Description of the Related Art

U.S. Pat. Nos. 6,096,052 and 6,432,116 each entitled “Occluding Device and Method of Use” describe a device for occluding a body lumen. Each of the noted U.S. patents is hereby incorporated by reference. The '052 and '116 patents describe a contraceptive or sterilization device for occluding a reproductive tract or lumen to prevent the passage of reproductive cells through the tract or lumen. The device generally comprises a tubular member and a mesh member, which is transversely disposed on the tubular member. The mesh member is permeable to allow for tissue ingrowth, which produces a tissue impregnated mesh occluding the body lumen. The occluding device of the invention can be used in the fallopian tubes of a female patient, the vas deferens of a male patient, or other body lumen. The device can additionally be used for delivering drugs or other substances to the bodies of human or animal subjects.

The device is delivered into the lumen of an anatomical passageway via use of an endoscope and, in the instance of a fallopian tube, via the use of a hysteroscope. Hysteroscopes are used for in vivo procedures because they enable physicians to look and operate inside a patient's body using a small camera. Hysteroscopes generally have a working channel through which catheters and other delivery devices can be inserted to perform in vivo procedures. In the instance of positioning an occluding device within a fallopian tube, the tubal ostium of the uterus must be visualized with a flexible or rigid scope. The opposing walls of the uterus are normally pressed together. In order to separate the uterine walls, the physician must deliver fluid to the uterus under pressure through a port in the scope. Once the scope is in place, the physician must deliver the catheter or delivery device to the site of the in vivo procedure. This requires opening the working channel of the scope so the catheter or delivery device can be inserted therethrough. Because the fluid in the uterus is under pressure, if there is not a good seal at the opening of the working channel, fluid can escape through the working channel of the scope and spray outward into the room.

In response to this problem, scope manufacturers offer scope seals that are specifically sized for certain diameter catheters. Unfortunately, these seals are designed for a range of catheters and not a specific catheter such as the delivery catheter for the occluding device. Because of this, among other problems, physicians may have unexpected leaks, may need to pay more to have a proper seal for the delivery catheter, may not have a seal readily available to perform a particular procedure, and/or may cause damage to the delivery catheter due to using an improper seal. There exists a need for a delivery catheter for delivering occluding devices that includes a seal that will fit flexible and rigid hysteroscopes. There also exists a need for a seal that is specifically designed for the catheter to consistently provide a reliable seal, simplify the use of the catheter, reduce the risk of exposure to contaminated fluids and eliminate the need for purchasing and stocking additional components for performing in vivo procedures.

### BRIEF SUMMARY OF THE INVENTION

It is an object of the present invention to provide a system for performing a procedure in vivo, including a seal that fits flexible and rigid hysteroscopes. It is another object of the present invention to provide a seal that is specifically designed for the catheter to consistently provide a reliable seal. It is still another object of the present invention to provide a seal that simplifies the use of the catheter. It is yet another object of the present invention to provide a seal that reduces the risk of exposure to contaminated fluids. It is another object of the present invention to provide a seal that eliminates the need for purchasing and stocking additional components for performing in vivo procedures. Finally, it is an object of the present invention to provide a method for positioning the scope seal on a fitting of a hysteroscope.

These and other objects and advantages of the present invention are accomplished by the scope seal and method for sealing the working channel of a hysterscope in accordance with the present invention. The scope seal includes distal and proximal ends, a substantially hollow body longitudinally extending between the distal and proximal ends, a shaft seal transversely disposed across the proximal end, a catheter sealing mechanism configured to receive at least one catheter therethrough, and a chamber disposed within the body at the distal end, where the chamber is configured to receive a fitting on the working channel of the scope.

The system for performing a procedure in vivo includes a catheter having a proximal end, a distal end and a lumen extending in part therein, a hysteroscope having a working channel therethrough in which the catheter is inserted and an adapter for receiving pressurized inflation fluid, a fitting positioned on a proximal end of the hysteroscope, and a seal having distal and proximal ends, a substantially hollow body longitudinally extending between the distal and proximal ends, a shaft seal transversely disposed across the proximal end of the seal, a catheter sealing mechanism configured to receive at least one catheter therethrough, and a chamber disposed within the body at the distal end of the seal to receive the fitting on the proximal end of the hysteroscope.

The method for positioning a scope seal on a hysteroscope includes providing a catheter having a proximal end, a distal end and a lumen extending in part therein, providing a hysteroscope having a working channel therethrough, providing a fitting on a proximal end of the hysterscope for securing the catheter through the hysterscope, providing a seal at a distal end of the fitting, where the seal has distal and proximal ends, a substantially hollow body longitudinally extending between the distal and proximal ends, a shaft seal transversely disposed across the proximal end of the seal, a catheter sealing mechanism for receiving at least one catheter therethrough, and a chamber for receiving the fitting on said proximal end of the hysteroscope, inserting the catheter into the proximal end of the seal, through the shaft seal and the catheter sealing mechanism, inserting the catheter through the fitting and the working channel of the scope, and securing the seal on the fitting by sliding the chamber over a proximal end of the fitting.

The catheter sealing mechanism of the seal, system and method can be any type of catheter sealing means, including but not limited to duck bill valves, conic valves, and/or membranes. The invention will be further described with reference to the following detailed description taken in conjunction with the drawings.

### BRIEF DESCRIPTION OF THE DRAWINGS

FIG. 1 illustrates female reproductive anatomy, a hysteroscope with a delivery catheter, and an occluding device implantable within a fallopian tube.

FIG. 2 is an elevational view, partially in section, of a delivery catheter for delivering the occluding device.

FIG. 3 is an elevational view, partially in section, of a delivery catheter having an inflatable member for delivering the occluding device.

FIG. 4 is a side, sectional view of the scope seal of the present invention.

FIG. 5 is a perspective, sectional view of the scope seal of the present invention, including a pressure relief chamber.

FIG. 6 is a perspective, sectional view of the scope seal having a duckbill valve formed on a shaft seal at a proximal end of the hysteroscope seal.

FIG. 7 is a front view of a conic valve.

FIG. 8 is a side cross sectional view taken along line A-A of FIG. 7.

FIG. 9 is a rear view of the conic valve of FIG. 7.

FIG. 10 is a side cross sectional view of the conic valve of FIG. 7 showing the conic valve about a scope port and a catheter extending through the conic valve and into the scope port.

FIG. 11 is a front view of a membrane valve.

FIG. 12 is a side cross sectional view of the membrane valve taken along line A-A of FIG. 11.

FIG. 13 is a rear view of the membrane valve of FIG. 11.

### DETAILED DESCRIPTION OF THE INVENTION

The present invention includes a hysteroscope 10 having a seal 11 that is configured to work with a delivery catheter 31 to deliver an occluding device 12 to at least one of a patient's fallopian tubes 42 through the working channel of hysteroscope 10. Seal 11 in accordance with the present invention prevents pressurized fluid, which is used to separate the walls of the uterus, from leaking through the working channel of hysteroscope 10.

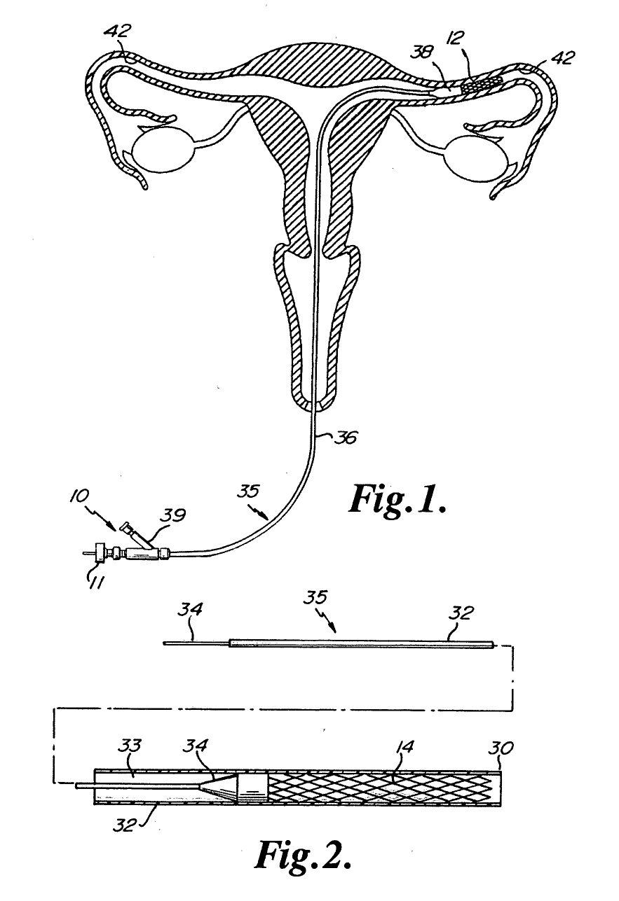

FIG. 1 illustrates the female reproductive anatomy, including fallopian tubes 42 in which the occluding device 12 is installed. In FIG. 1, occluding device 12 is mounted on an inflatable member 38 of a catheter 35 and positioned within at least one of the patient's fallopian tubes 42. Occluding device 12 includes a tubular member 14 having a relatively small transverse dimension, which is mounted on an exterior of inflatable member 38. In order to insert occluding device 12 within fallopian tube 42, catheter 35 is advanced, using visualization with hysterscope 10, through the working channel of hysteroscope 10. In so doing, catheter is inserted through seal 11 until tubular member 14 is positioned within at least one of the patient's fallopian tubes 42. Inflation fluid is introduced through an adapter 39 to expand inflatable member 38. Inflation of inflatable member 38 expands tubular member 14 to an open configuration, thereby lodging tubular member 14 in fallopian tube 42.

FIG. 2 illustrates one embodiment of delivery catheter 35 useful in the delivery of occluding device 12, particularly when occluding device 12 has a self expanding tubular member 14. Delivery catheter 35 includes an elongated shaft 32 having a lumen 33 extending therein. Self expanding tubular member 14 may be deformed into the smaller diameter configuration within lumen 33 of delivery catheter 35, and expanded into the larger diameter configuration within the patient's body lumen by longitudinally displacing tubular member 14 out a distal end 30 of delivery catheter 35 to thereby remove the radially compressive force exerted on tubular member 14 by delivery catheter 35.

A pusher 34 can be slidably received within lumen 33 of delivery catheter 35. Pusher 34 can be used to longitudinally displace tubular member 14 out distal end 30 of delivery catheter 35.

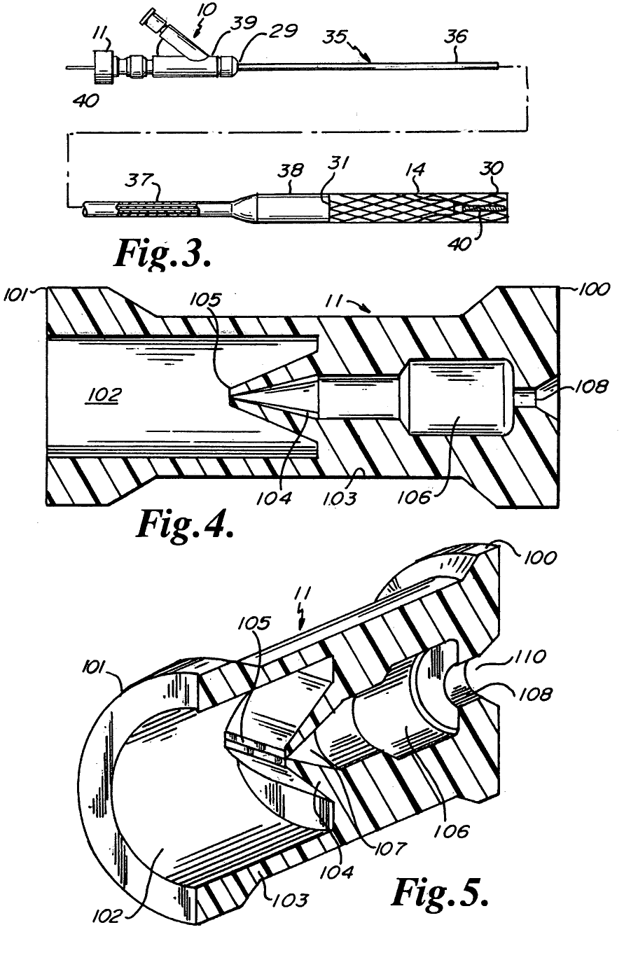

In another embodiment, tubular member 14 can be expanded using catheter 35 with inflatable member 38 being inserted into an open second end 31 of tubular member 14. Referring now to FIG. 3, there is shown catheter 35 being passed through the working port of hysteroscope 10 and seal 11 of the present invention. Catheter 35 is useful in positioning and implanting occluding device 12 within a patient's body lumen. Cather 35 includes an elongated shaft 36 having an inflation lumen 37 which is in fluid communication with inflatable member 38 mounted on a distal end 30 of elongated shaft 36 and an adapter 39 on a proximal end 29 of elongated shaft 36. Tubular member 14 is mounted on inflatable member 38, and is configured to closely conform to the diameter of inflatable member 38 in the uninflated position thereby facilitating introduction of tubular member 14 into the desired body lumen. Tubular member 14 may be deformed to facilitate mounting onto inflatable member 38. Tubular member 14 can then be expanded by inflatable member 38 to an open, expanded configuration thereby lodging tubular member within the patient's body lumen. A guidewire 40 within lumen 37 of elongated shaft 36 may extend through tubular member 14 to facilitate positioning of tubular member 14 within the patient's body lumen.

Seal 11 has a proximal end 100 through which catheter 35 can be received and a distal end 101 that communicates with a fitting on hysteroscope 10. A substantially hollow body 103 extends longitudinally between proximal end 100 and distal end 101 of seal 11. Seal 11 also includes a catheter sealing mechanism positionable within body 103, a shaft seal 108, a chamber 102 and a pressure relief chamber 106. Catheter sealing mechanism can be any type of catheter sealing means, including but not limited to a duckbill valve 104, a conic valve 112, a membrane 128, and the like, as discussed in greater detail below. While these three embodiments are depicted in the figures and discussed herein, those skilled in the art can appreciate that various designs of catheter sealing mechanisms can be used to provide support of catheter 35 within seal 11 and to provide a sealing point between pressurized fluid in the working channel of hysteroscope 10 and the physician.

Referring now to FIGS. 4 and 5, there is shown one embodiment of seal 11 in accordance with the present invention. Seal 11 includes a duckbill valve 104, a shaft seal 108, a chamber 102 and a pressure relief chamber 106. Seal 11 can be configured as a single piece silicone seal.

Chamber 102 is formed in the interior portion of distal end 101 of seal 11 and communicates with a fitting in the working channel of hysteroscope 10 (shown in FIGS. 1 and 3). Chamber 102 in the embodiment depicted in FIGS. 4 and 5 is configured to communicate with a luer type fitting on the working channel of hysteroscope 10. Those skilled in the art can appreciate that chamber 102 can be configured in various shapes and sizes such that it communicates with various fittings placed on the working channel of hysteroscope 10.

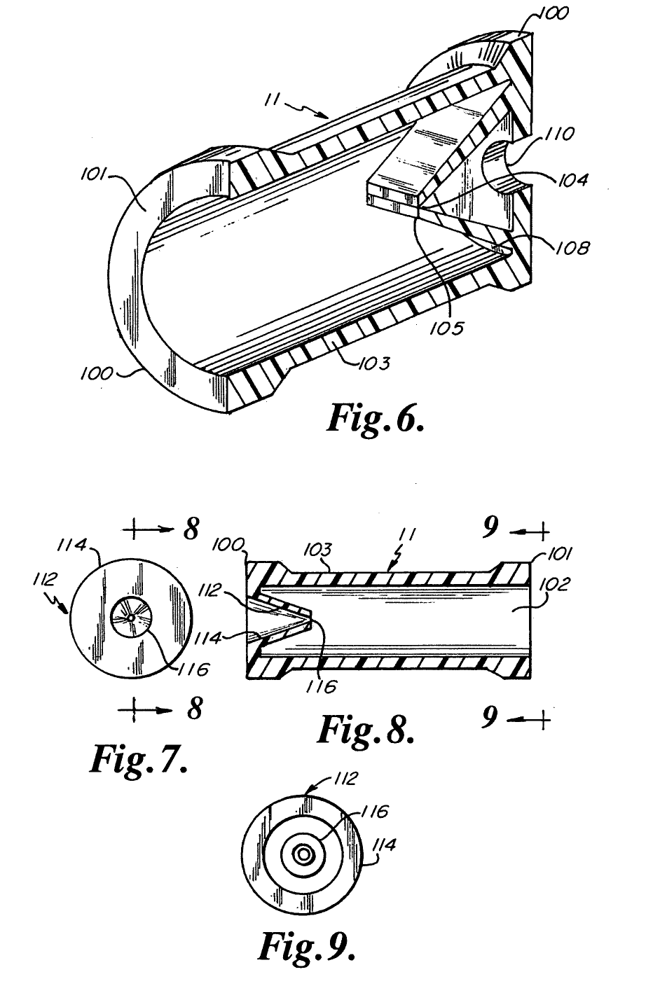

Referring still to FIGS. 4 and 5, in one embodiment, pressure relief chamber 106 is an open section generally positioned at proximal end 100 of seal between duckbill valve 104 and shaft seal 108. Referring now to FIG. 6, in another embodiment, duckbill valve 104 is formed directly on shaft seal 108 such that any fugitive pressurized fluid can be trapped between the end of a fitting positioned within chamber 102 and shaft seal 108 and around the perimeter of duckbill valve 104.

Referring to FIGS. 4-6, shaft seal 108 is transversely positioned on proximal end 100 of seal 11 with at least one opening 110 sized to fit around an outer diameter of catheter 35. Shaft seal 108 can be configured of silicone. Those skilled in the art can appreciate that shaft seal 108 can have various shapes and/or sizes. Shaft seal 108 can also have at least one opening 110 that is sized and shaped such that various sizes of catheters 35 can be inserted therethrough. For example, shaft seal 108 can include gripping extensions or similar such extensions that can be used to provide a longer section of sealing along the portion of catheter 35 adjacent seal 11. Alternately, shaft seal 108 can include deformable and/or memory shape materials for providing a seal for catheters 35 having various diameters and being constructed of various materials.

Duckbill valve 104 can have at least one cross slit 105 enabling catheter 35 to be passed therethrough. Duckbill valve 104 can have one horizontal cross slit 105 (as shown in FIG. 6) and/or one vertical cross slit 107. FIG. 5 depicts duckbill valve 104 having both a horizontal cross slit 105 and a vertical cross slit 107. Those skilled in the art can appreciate that other types of valves or pass through components can be used as will be discussed in greater detail below.

Referring now to FIGS. 7-9, in another embodiment of the present invention, the catheter sealing mechanism of seal 11 is a conic valve 112. Conic valve 112 has an external opening 114 on proximal end 100 of seal 11 and an internal opening 116 within body 103 of seal 11. External opening 114 has a larger outer diameter than internal opening 116, thereby creating a conic shape.

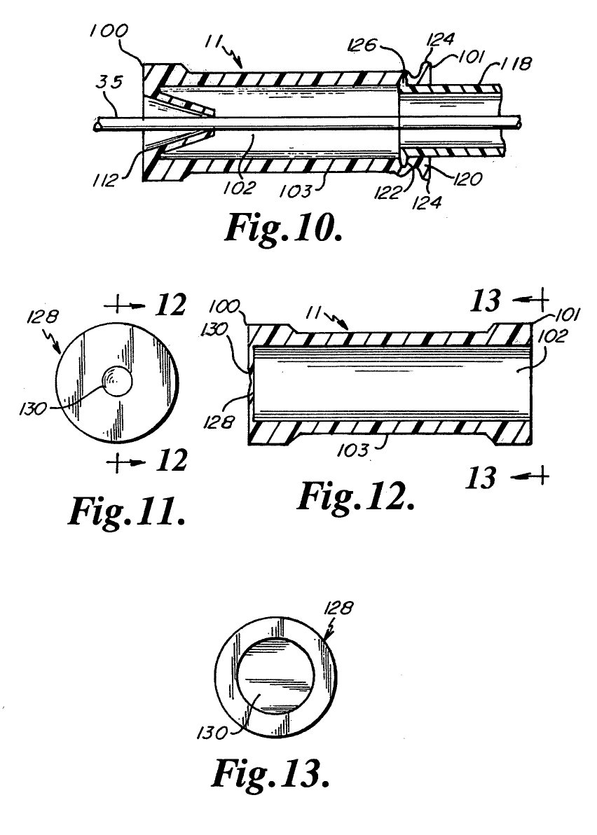

Referring now to FIG. 10, conic valve 112 is shown about a scope port 118 and catheter 35 extending through conic valve 112 and into scope. Catheter 35 is inserted first into external opening 114 and then into internal opening 116 such that fugitive pressurized fluid can be trapped in pressure relief chamber 102 located between conic valve 112 and scope port 118. In this embodiment, a scope seal 120 can be included in seal 11. Scope seal 120 has a recessed portion 122 and a radially extending portion 124. Scope port 118 can be slid into scope seal 120 such that radially extending portion 124 of scope seal 120 is overlaps scope seal 118 and an annular lip 126 on scope port 118 received within recessed portion 122 of scope seal 120.

Referring now to FIGS. 11-13, in yet another embodiment of the present invention, the catheter sealing mechanism of seal 11 includes a membrane 128 transversely disposed across proximal end 100 of seal 11. Membrane 128 includes a notched portion 130 such that membrane 128 can be pierced with catheter 35 before catheter 35 can be pushed through seal 11. While membrane 128 depicted in FIGS. 11-13 includes notched area 130, those skilled in the art can appreciate that membrane 128 could include a slit, hole, or other such opening to facilitate insertion of catheter 35 therethrough. Alternately, membrane 128 could be formed without a notched area 130 or any opening and still allow for piercing and insertion of catheter 35 therethrough. The embodiment depicted in FIGS. 11-13 also includes pressure relief chamber 102 within body 103 of seal 11 for trapping fugitive pressurized fluid between membrane 128 and a fitting on the working channel of hysteroscope 10.

In operation, chamber 102 slips on to the working channel of hysteroscope 10. Shaft seal 108 seals around catheter 35 on initial penetration creating a barrier between the physician and the catheter sealing mechanism. Further penetration of catheter 35 causes catheter 35 to break through catheter sealing mechanism allowing some release of saline into pressure relief chamber 106 until catheter sealing mechanism forms around catheter 35 thereby preventing fugitive pressurized fluid from spraying or running out of seal 11.

It should be noted that while the scope seal of the present invention has been described in reference to its use with a hysteroscope it is understood that the seal may be equally useful on other types of endoscopes and should not be construed as having a use limited to a hysteroscope.

The present invention may be embodied in other specific forms without departing from the spirit of the essential attributes thereof; therefore, the illustrated embodiments should be considered in all respects as illustrative and not restrictive.

What is claimed is:

1. A scope seal comprising:
distal and proximal ends;
a substantially hollow body longitudinally extending between said distal and proximal ends;
a shaft seal transversely disposed across said proximal end;
a catheter sealing mechanism configured to receive at least one catheter therethrough; and
a chamber disposed within said body at said distal end, said chamber configured to receive a fitting on a working channel of a scope.

2. The scope seal of claim 1, further comprising a pressure relief chamber between said catheter sealing mechanism and said shaft seal.

3. The scope seal of claim 2 wherein said catheter sealing mechanism is a duck bill valve disposed within said body adjacent said pressure relief chamber.

4. The scope seal of claim 3 said duck bill valve including a horizontal cross slit configured to accommodate said at least one catheter therethrough.

5. The scope seal of claim 3 said duck bill valve including a vertical cross slit configured to accommodate said at least one catheter therethrough.

6. The scope seal of claim 3 said duck bill valve including a vertical cross slit and a horizontal cross, both cross slits being configured to accommodate said at least one catheter therethrough.

7. The scope seal of claim 1 wherein said catheter sealing mechanism is a conic valve.

8. The scope seal of claim 1 wherein said catheter sealing mechanism is a membrane.

9. The scope seal of claim 8 said membrane having a notch configured to facilitate insertion of said at least one catheter therethrough.

10. A system for performing a procedure in vivo comprising:
a catheter having a proximal end, a distal end and a lumen extending in part therein;
a hysteroscope having a working channel therethrough in which said catheter is inserted and an adapter configured to receive pressurized inflation fluid;
a fitting positioned on a proximal end of said hysteroscope; and
a seal having distal and proximal ends, a substantially hollow body longitudinally extending between said distal and proximal ends, a shaft seal transversely disposed across said proximal end of said seal, a catheter sealing mechanism configured to receive at least one catheter therethrough, and a chamber disposed within said body at said distal end of said seal to receive said fitting on said proximal end of said hysteroscope.

11. The system of claim 10 said seal further comprising a pressure relief chamber between said catheter sealing mechanism and said shaft seal.

12. The system of claim 11 wherein said catheter sealing mechanism is a duck bill valve disposed within said body adjacent said pressure relief chamber.

13. The system of claim 12 said duck bill valve including a horizontal cross slit configured to accommodate said at least one catheter therethrough.

14. The system of claim 12 said duck bill valve including a vertical cross slit configured to accommodate said at least one catheter therethrough.

15. The system of claim 12 said duck bill valve including a vertical cross slit and a horizontal cross, both cross slits being configured to accommodate said at least one catheter therethrough.

16. The system of claim 10 wherein said catheter sealing mechanism is a conic valve.

17. The system of claim 10 wherein said catheter sealing mechanism is a membrane.

18. The system of claim 17 said membrane having a notch configured to facilitate insertion of said at least one catheter therethrough.

19. A scope seal comprising:
distal and proximal ends;
a substantially hollow body longitudinally extending between said distal and proximal ends;
a shaft seal transversely disposed across said proximal end;
a catheter sealing means for receiving at least one catheter therethrough and providing a seal around said catheter to prevent escape of said fluid around said catheter; and
a chamber disposed within said body at said distal end, said chamber configured to receive a fitting on a working channel of a scope.

20. The scope seal of claim 19, further comprising a pressure relief chamber between said catheter sealing means and said shaft seal.

21. The scope seal of claim 20 wherein said catheter sealing means is a duck bill valve disposed within said body adjacent said pressure relief means.

22. The scope seal of claim 21 said duck bill valve including a horizontal cross slit configured to accommodate said at least one catheter therethrough.

23. The scope seal of claim 21 said duck bill valve including a vertical cross slit configured to accommodate said at least one catheter therethrough.

24. The scope seal of claim 21 said duck bill valve including a vertical cross slit and a horizontal cross, both cross slits being configured to accommodate said at least one catheter therethrough.

25. The scope seal of claim 19 wherein said catheter sealing means is a conic valve.

26. The scope seal of claim 19 wherein said catheter sealing means is a membrane.

27. The scope seal of claim 26 said membrane having a notch configured to facilitate insertion of said at least one catheter therethrough.

28. A method for positioning a scope seal on a hysteroscope comprising:
providing a catheter having a proximal end, a distal end and a lumen extending in part therein;
providing a hysteroscope having a working channel therethrough;
providing a fitting on a proximal end of said hysterscope for securing said catheter through said hysterscope;
providing a seal at a distal end of said fitting, wherein said seal has distal and proximal ends, a substantially hollow body longitudinally extending between said distal and proximal ends, a shaft seal transversely disposed across said proximal end of said seal, a catheter sealing mechanism for receiving at least one catheter therethrough, and a chamber for receiving said fitting on said proximal end of said hysteroscope;
inserting said catheter into said proximal end of said seal, through said shaft seal and said catheter sealing mechanism;
inserting said catheter through said fitting and said working channel of said scope; and
securing said seal on said fitting by sliding said chamber over a proximal end of said fitting.

29. The method of claim 28, further comprising a pressure relief chamber between said catheter sealing mechanism and said shaft seal.

30. The method of claim 29 wherein said catheter sealing mechanism is a duck bill valve disposed within said body adjacent said pressure relief chamber.

31. The method of claim 30 said duck bill valve including a horizontal cross slit configured to accommodate said at least one catheter therethrough.

32. The method of claim 30 said duck bill valve including a vertical cross slit configured to accommodate said at least one catheter therethrough.

33. The method of claim 30 said duck bill valve including a vertical cross slit and a horizontal cross, both cross slits being configured to accommodate said at least one catheter therethrough.

34. The method of claim 28 wherein said catheter sealing mechanism is a conic valve.

35. The method of claim 28 wherein said catheter sealing mechanism is a membrane.

36. The method of claim 35 wherein said membrane has a notch configured to facilitate insertion of said at least one catheter therethrough.

37. A scope seal comprising:
distal and proximal ends having a substantially hollow body longitudinally extending therebetween;
a shaft seal transversely disposed across said proximal end;
a catheter sealing mechanism configured to receive at least one catheter therethrough, said catheter sealing mechanism is selected from the group consisting of duck bill valves, conic valves and membranes;
a chamber disposed within said body at said distal end, said chamber configured to receive a fitting on a working channel of a scope; and a pressure relief chamber between said catheter sealing mechanism and said shaft seal.

38. A system for performing a procedure in vivo comprising:
a catheter having a proximal end, a distal end and a lumen extending in part therein;
a hysteroscope having a working channel therethrough in which said catheter is inserted and an adapter configured to receive pressurized inflation fluid;
a fitting positioned on a proximal end of said hysteroscope; and
a seal having distal and proximal ends, a substantially hollow body longitudinally extending between said distal and proximal ends, a shaft seal transversely disposed across said proximal end of said seal, a catheter sealing mechanism configured to receive at least one catheter therethrough, said catheter sealing mechanism is selected from the group consisting of duck bill valves, conic valves and membranes, and a chamber disposed within said body at said distal end of said seal to receive said fitting on said proximal end of said hysteroscope.

39. A method for positioning a scope seal on a hysteroscope comprising:
providing a catheter having a proximal end, a distal end and a lumen extending in part therein;
providing a hysteroscope having a working channel therethrough;
providing a fitting on a proximal end of said hysterscope for securing said catheter through said hysterscope;
providing a seal at a distal end of said fitting, wherein said seal has distal and proximal ends, a substantially hollow body longitudinally extending between said distal and proximal ends, a shaft seal transversely disposed across said proximal end of said seal, a catheter sealing mechanism for receiving at least one catheter therethrough, said catheter sealing means is selected from the group consisting of duck bill valves, conic valves and membranes, and a chamber for receiving said fitting on said proximal end of said hysteroscope;
inserting said catheter into said proximal end of said seal, through said shaft seal and said catheter sealing mechanism;
inserting said catheter through said fitting and said working channel of said scope; and
securing said seal on said fitting by sliding said chamber over a proximal end of said fitting.
